(19) United States
(12) Patent Application Publication
(10) Pub. No.: US 2024/0176865 A1
(43) Pub. Date: May 30, 2024

(54) SURGICAL ROBOT AND SURGICAL SYSTEM

(71) Applicant: Sony Group Corporation, Tokyo (JP)

(72) Inventor: Hiroyuki SUZUKI, Tokyo (JP)

(73) Assignee: Sony Group Corporation, Tokyo (JP)

(21) Appl. No.: 18/551,602

(22) PCT Filed: Dec. 28, 2021
(86) PCT No.: PCT/JP2021/048989

§ 371 Date: Sep. 21, 2023

(30) Foreign Application Priority Data
Mar. 31, 2021 (JP) 2021-060152

(51) Int. Cl. G06F 21/44; A61B 34/37; A61B 90/00; A61B 90/90; A61F 9/007; G16H 40/63

(52) U.S. Cl. CPC G06F 21/44; A61B 34/37; A61B 90/08; A61B 90/90; A61F 9/007; G16H 40/63; A61B 2090/0803

(57) ABSTRACT

Provided is a surgical robot that spatially separates non-clean areas by disposable and facilitates setup.

A surgical robot is equipped with a robot arm to which a surgical tool is mounted; and a storage circuit that stores information associated with motion control of the robot arm. The storage circuit stores at least one of a robot ID for identifying an individual or a model of the surgical robot, configuration information of the robot arm, or calibration information for when controlling the motion of the robot arm. In addition, the storage circuit is writable and stores information for identifying presence or absence of use or operation used.

### TECHNICAL FIELD

The technology disclosed in the present specification (hereinafter referred to as “the present disclosure”) relates to a surgical robot and a surgical system used in a surgical operation.

### BACKGROUND ART

Surgical tools used in a surgical operation need to be kept clean. Therefore, in a case where the surgical tool is reused, cleaning and sterilization treatment need to be repeated for each use. However, although inactivation and removal of bacteria, viruses, and the like can be performed by the cleaning and sterilization treatment, there is a problem that protein remains. Furthermore, repetitive sterilization also increases the risk of failure. Handling of expensive and fragile surgical tools is cumbersome for operators and nurses.

Recently, a robotics technology has also been introduced into the medical field, and a master-slave system surgical robot is used to perform operation (see e.g., Patent Document 1). In a surgical operation using a robot, a clean area and an unclean area of a robot arm need to be spatially separated. In most cases, a drape is mounted between the surgical tool and the robot arm to separate the clean area and the unclean area, but the setup is complex as the drape is bulky, and in addition, care must be taken so as not to touch the drape at all times in the operating room.

### CITATION LIST

### Patent Document

Patent Document 1: WO 2019/012812

### SUMMARY OF THE INVENTION

### Problems to be Solved by the Invention

An object of the present disclosure is to provide a surgical robot and a surgical system that are used in a surgical operation and realize spatial separation between a clean area and an unclean area.

### Solutions to Problems

The present disclosure has been made in view of the above problems, and a first aspect thereof relates to a surgical robot equipped with:
a robot arm to which a surgical tool is mounted; and
a storage circuit that stores information associated with motion control of the robot arm.

The storage circuit stores at least one of a robot ID for identifying an individual or a model of the surgical robot, configuration information of the robot arm, or calibration information for when controlling the motion of the robot arm. In addition, the storage circuit is writable and stores information for identifying presence or absence of use or operation used.

Furthermore, the surgical tool is loaded with a storage circuit. The storage circuit loaded on the surgical tool stores at least one of a surgical tool ID for identifying an individual or a model of the surgical tool or calibration information for when operating the surgical tool. Furthermore, the storage circuit loaded on the surgical tool is writable, and stores at least one of presence or absence of use, information for specifying a patient on which the surgical tool is used, or information for identifying operation using the surgical tool.

Furthermore, a second aspect of the present disclosure relates to a surgical system including:
a surgical robot including a robot arm to which a surgical tool is mounted and a storage circuit that stores information associated with motion control of the robot arm; and
a control unit that controls the surgical robot on the basis of the information read from the storage circuit.

However, the term, “system”, as used herein refers to a logical assembly of multiple devices (or functional modules that implement specific functions), and each of the devices or functional modules may be or may be not in a single housing.

### Effects of the Invention

According to the present disclosure, a surgical robot that is used in a surgical operation, spatially separates an unclean area by being disposable, and facilitates setup, and a surgical system that uses a disposable surgical robot can be provided.

Note that, the effect described in this specification is illustrative only and the effect by the present invention is not limited to this. Furthermore, there also is a case in which the present disclosure further has an additional effect in addition to the above-described effect.

Still another object, feature, and advantage of the present disclosure will become clear by further detailed description with reference to an embodiment to be described later and the attached drawings.

### BRIEF DESCRIPTION OF DRAWINGS

FIG. 1 is a diagram illustrating a functional configuration example of a surgical system 100.

FIG. 2 is a diagram illustrating a configuration example of a disposable surgical robot 200.

FIG. 3 is a diagram illustrating a layout of an eyeground operation using the surgical robot 200.

FIG. 4 is a diagram illustrating a state in which a surgical tool mounted to the surgical robot 200 is replaced.

FIG. 5 is a diagram illustrating a configuration example of an authentication system 500.

FIG. 6 is a flowchart illustrating a processing procedure executed in a surgical system 100 using a disposable surgical robot and surgical tool.

FIG. 7 is a diagram illustrating a cross-sectional configuration example of an electric circuit board 700 applied to a multi-link structure.

FIG. 8 is a diagram illustrating a configuration example of an open link structure 800 configured using an FPC.

FIG. 9 is a diagram illustrating an example of a closed link structure 900 configured using an FPC.

FIG. 10 is a diagram illustrating a configuration example of a surgical robot 1000 configured with a closed link structure using an FPC.

FIG. 11 is a diagram illustrating a configuration of a degree of freedom of the surgical robot 1000.

FIG. 12 is a diagram illustrating an operation example of the surgical robot 1000.

FIG. 13 is a diagram illustrating a three-dimensional image example of the surgical robot 1000 configured with the closed link structure using an FPC.

### MODE FOR CARRYING OUT THE INVENTION

Hereinafter, the present disclosure will be described in the following order with reference to the drawings.
A. Overview
B. Surgical system
C. Configuration of surgical robot
D. Replacement of surgical tool
E. Authentication process
F. Processing operation of surgical system
G. Implementation example of surgical robot
H. Effects

### A. Overview

In general, surgical operation is a difficult task performed by the operator's sensorimotor. In particular, in the case of operation using a fine surgical tool under a small and fragile environment, such as ophthalmic operation, the operator needs to suppress tremor of the hand and perform a micron-order operation. Therefore, in order to suppress tremor of a hand of an operator and absorb a difference in skill between operators by operational support, a surgical robot is becoming popular.

In the case of a surgical operation using a surgical robot, when an attempt is made to separate a clean area and an unclean area using a drape, setup is complex, and care needs to be taken so as not to touch the drape during the operation. In addition, there is a problem that a component for separating an unclean area such as a drape inhibits the field of view of the microscope or collides with another surgical tool. Furthermore, in ophthalmic operation, operation is performed in an extremely short time as compared to other diagnosis and treatment departments, but the time required for setup is a bottleneck for reducing the total operation time. In addition, in a case where the surgical tool mounted to the surgical robot is reused, cleaning and sterilization treatment are repeated for each use, and thus, there is a problem such as residual of protein and an increase in the risk of failure.

Therefore, the present disclosure proposes a surgical robot that is used for, for example, ophthalmic operation and can be made disposable for each operation.

In a case where a disposable surgical robot is used, a new surgical robot is installed in an operating room for each operation, and thus the setup of the surgical robot needs to be facilitated. Thus, the surgical robot according to the present disclosure is loaded with a storage circuit that stores, for example, information necessary for motion control of the robot arm. Therefore, on the control system side that controls the surgical robot, the surgical robot can be easily set up by reading the necessary information from the storage circuit loaded on the surgical robot each time the surgical robot is newly installed. Note that the information necessary for the motion control of the robot arm includes a robot ID for identifying an individual or a model of the surgical robot, robot configuration information such as number of inks and number of joints, calibration information for when controlling the motion of the robot arm, and the like.

In addition, the disposable surgical robot needs to be reliably discarded after being used for operation once and managed so as not to be used again by mistake. Therefore, the surgical robot may be loaded with a writable storage circuit, and information for identifying the presence or absence of use or the operation used may be stored in the storage circuit. In such a case, the control system can access the storage circuit at the time of setting up the surgical robot to confirm that the surgical robot is unused. Then, when the used surgical robot is erroneously installed, the control system may issue a warning to urge disposal of the surgical robot and installation of a new surgical robot, or restrict the operation of the surgical robot. Note that information including a combination of presence or absence of use, an operation ID for identifying operation, an operating room ID for identifying an operating room, and a date and time on which operation is performed may be stored in the storage circuit. Alternatively, a simple use flag indicating the presence or absence of use may be stored in the storage circuit.

For example, a surgical robot used for laparoscopic operation or the like is required to have a wide movable range, which inevitably increases the size of the robot arm, and accordingly, the number of parts and device cost increase, and hence it is economically difficult to make the robot arm disposable, and the burden of disposal work is large. On the other hand, since the surgical robot used for ophthalmic operation may have a small movable range, the robot arm is small in size, and the number of parts and device cost can be reduced, and hence even if the robot arm is made disposable, an increase in operation cost is suppressed, and the burden of disposal work is reduced. Therefore, it can be said that the present disclosure is more preferably applied to a surgical robot for surgical operation.

Furthermore, in the present disclosure, in addition to the surgical robot, a surgical tool mounted to the surgical robot is also made disposable, thus freeing from problems such as a risk of failure due to repetition of cleaning and sterilization treatment and remaining of protein.

It is assumed that the surgical tool mounted to the surgical robot is replaced in one operation. On the control system side that controls the surgical robot, it is necessary to acquire and set up information necessary for operating the surgical tool through the surgical robot each time the surgical tool mounted to the surgical robot is replaced. Therefore, in the present disclosure, the surgical tool is loaded with a storage circuit that stores necessary information, and the surgical robot transmits information read from the storage circuit of the surgical tool being mounted to the control system side. Therefore, the control system can easily set up the surgical robot and the surgical tool and immediately start the surgical tool operation. Note that the storage circuit of the surgical tool includes a surgical tool ID for identifying an individual or model of the surgical tool, calibration information when operating the surgical tool (e.g., when opening/closing the forceps), and the like.

In addition, it is assumed that a surgical tool used by being attached to the surgical robot is removed from the surgical robot in the middle and then used again on the same surgical robot (or another robot) in one operation. This is because there is no problem such as infection of bacteria if the patients are the same. Therefore, the surgical tool may be loaded with a writable storage circuit, and the storage circuit may store presence or absence of use, a patient ID for specifying a patient who has used the surgical tool, and an operation ID for identifying operation (or may be a combination of an operating room ID for identifying an operating room and a date and time on which an operation is performed). In such a case, the control system can access the storage circuit of the surgical tool mounted to the surgical robot to check whether the surgical tool is unused, whether the surgical tool is used again in one operation, whether the surgical tool is used again for the same patient, and the like. Then, when the surgical tool used in another operation is mounted to the surgical robot by mistake, the control system may issue a warning to urge replacement with a new surgical tool or restrict the operation of the surgical robot.

### B. Surgical System

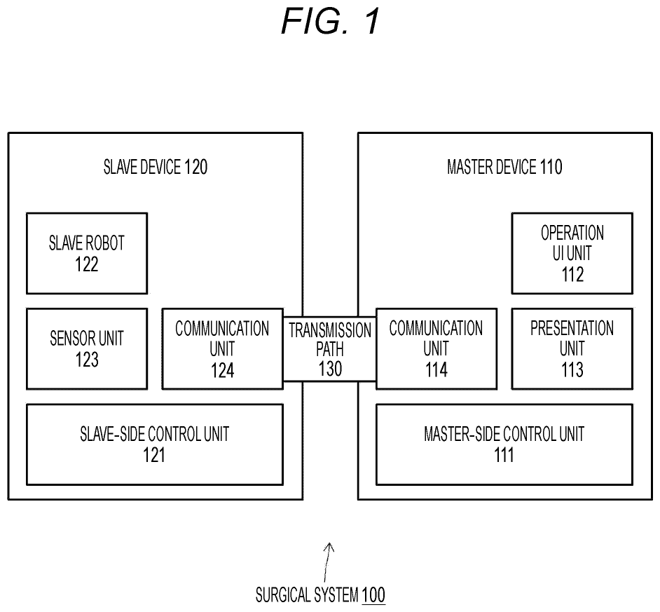

FIG. 1 schematically illustrates a functional configuration example of a surgical system 100 to which the present disclosure is applied. The surgical system 100 is a master-slave system in which a user such as an operator performs an operation on the master side and controls driving of a robot according to the operation of the user on the slave side to perform operation.

The illustrated surgical system 100 includes a master device 110 in which a user (operator) instructs work such as operation and a slave device 120 that performs the operation according to an instruction from the master device 110. Retinal operation is mainly assumed for the operation referred to here. The master device 110 and the slave device 120 are interconnected via a transmission path 130. The transmission path 130 is desirably capable of performing signal transmission with a low delay using, for example, a medium such as an optical fiber.

The master device 110 includes a master-side control unit 111, an operation user interface (UI) unit 112, a presentation unit 113, and a master-side communication unit 114. The master device 110 operates under the overall control of the master-side control unit 111.

The operation UI unit 112 includes a device for a user (operator etc.) to input an instruction to a slave robot 112 (described later) that operates a surgical tool such as forceps in the slave device 120. The operation UI unit 112 includes, for example, a dedicated input device such as a controller or a joystick, and a general-purpose input device such as a GUI screen for inputting a mouse operation or a touch operation of a fingertip.

The presentation unit 113 presents information associated with an operation performed by the slave device 120 to a user (operator) operating the operation UI unit 112 on the basis of sensor information mainly acquired by a sensor unit 123 (described later) on the slave device 120 side.

For example, in a case where the sensor unit 123 on the slave device 120 side is equipped with an RGB camera or an optical coherence tomography (OCT) that captures a microscopic image in which the surface of the affected part is observed, or is equipped with an interface that takes in an imaged image (hereinafter, simply referred to as a “microscopic image”) or an OCT image of the RGB camera, and these image data are transferred to the master device 110 via the transmission path 130 with a low delay, the presentation unit 113 displays a real-time microscopic image or OCT image of the affected part on the screen using a monitor display or the like.

Furthermore, in a case where the sensor unit 123 is equipped with a function of measuring an external force and a moment acting on the surgical tool operated by the slave robot 112, and such force sense information is transferred to the master device 110 with a low delay via the transmission path 130, the presentation unit 113 performs force sense presentation to the user (operator). For example, the presentation unit 113 may perform force sense presentation to the user (operator) using the operation UI unit 112.

The master-side communication unit 114 performs a signal transmission/reception process with the slave device 120 via the transmission path 130 under the control of the master-side control unit 111. For example, in a case where the transmission path 130 includes an optical fiber, the master-side communication unit 114 includes an electro-optical conversion unit that converts an electrical signal transmitted from the master device 110 into an optical signal, and a photoelectric conversion unit that converts an optical signal received from the transmission path 130 into an electrical signal. The master-side communication unit 114 transfers an operation command for the slave robot 122 input by the user (operator) via the operation UI unit 112 to the slave device 120 via the transmission path 130. Furthermore, the master-side communication unit 114 receives the sensor information transmitted from the slave device 120 via the transmission path 130.

On the other hand, the slave device 120 includes a slave-side control unit 121, a slave robot 122, a sensor unit 123, and a slave-side communication unit 124. The slave device 120 operates in accordance with an instruction from the master device 110 under the overall control of the slave-side control unit 121.

The slave robot 122 is, for example, an arm type surgical robot having a multi-link structure, and is loaded with a surgical tool such as forceps, as an end effector, at the tip (or the distal end). The slave-side control unit 121 interprets the operation command transmitted from the master device 110 via the transmission path 130, converts the operation command into a drive signal of an actuator that drives the slave robot 122, and outputs the drive signal. The slave robot 122 then operates on the basis of the drive signal from the slave-side control unit 121.

The slave robot 122 is assumed to perform retinal operation, for example, and is assumed to have a remote center of motion (RCM) structure. The RCM structure is a structure in which a rotation center (i.e., the remote rotation center) is disposed at a position away from a rotation center of a drive mechanism such as a motor to realize a pivot (fixed point) motion. The RCM structure is highly safe because a structure that always passes through the position (e.g. trocar position) of a hole drilled in the patient's body during the operation can be realized.

The sensor unit 123 includes a plurality of sensors for detecting a status in an affected area of the operation performed by the slave robot 122 or the slave robot 122, and further includes an interface for taking in sensor information from various sensor devices installed in the operating room. For example, the sensor unit 123 includes a force torque sensor (FTS) for measuring an external force and a moment acting during the operation on a surgical tool loaded at the tip (distal end) of the slave robot 122. In addition, the sensor unit 123 is provided with an interface through which the slave robot 122 takes in a microscopic image of the surface of an affected part during the operation or an OCT image in which a cross-section of the affected part (eyeball) is scanned.

The slave-side communication unit 124 performs a signal transmission/reception process with the master device 110 via the transmission path 130 under the control of the slave-side control unit 121. For example, in a case where the transmission path 130 includes an optical fiber, the slave-side communication unit 124 includes an electro-optical conversion unit that converts an electrical signal transmitted from the slave device 120 into an optical signal, and a photoelectric conversion unit that converts an optical signal received from the transmission path 130 into an electrical signal. The slave-side communication unit 124 transfers the force sense data of the surgical tool acquired by the sensor unit 123, the microscopic image of the affected part, the OCT image in which the cross-section of the affected part is scanned, and the like to the master device 110 via the transmission path 130. Furthermore, the slave-side communication unit 124 receives the operation command on the slave robot 122 transmitted from the master device 110 via the transmission path 130.

### C. Configuration of Surgical Robot

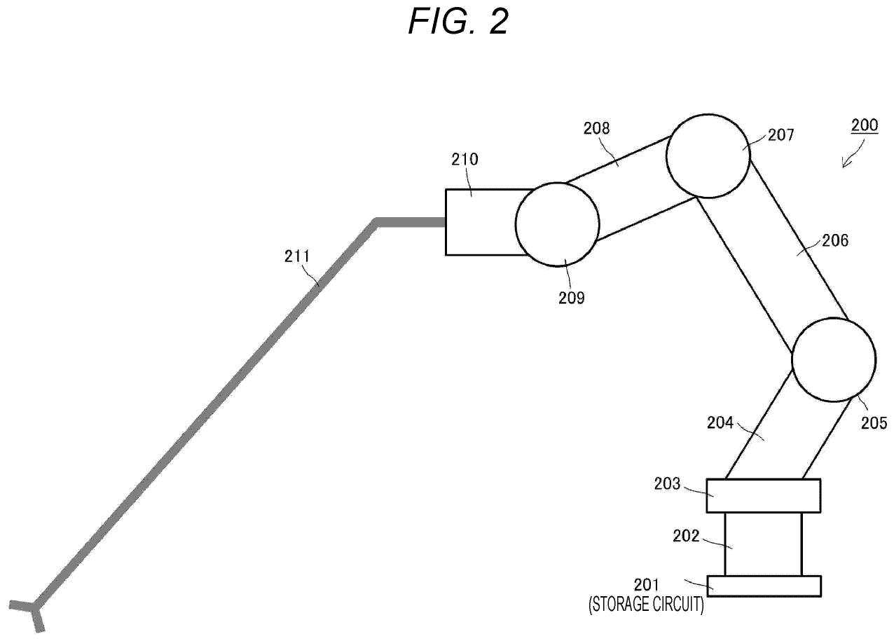

FIG. 2 schematically illustrates a configuration example of a disposable surgical robot 200 in the slave robot 122. The illustrated surgical robot 200 includes an interface unit 201 attached to the slave device 120, a link 202 attached perpendicularly to the interface unit 201, and a robot arm attached to the upper end of the link 202 by way of a joint 203. It is assumed that the joint 203 has a rotational degree of freedom about the yaw axis.

The robot arm has a serial link structure, and includes links 204, 206, 208, and 210, a joint 205 that hinge-couples the link 204 and the link 206, a joint 207 that hinge-couples the link 206 and the link 208, and a joint 209 that hinge-couples the link 208 and the link 210. Each of the joints 205, 207, and 209 has a rotational degree of freedom about the roll axis (or about an axis orthogonal to the yaw axis). Then, the surgical tool 211 is mounted to the link 210 at the distal end. The surgical tool 211 is replaceable. In addition, it is assumed that a mounting detection mechanism that detects whether or not the surgical tool 211 is mounted is provided.

The interface unit 201 includes a storage circuit accessible from the slave device 120 side. The storage circuit stores information necessary for motion control of the robot arm. Note that the information necessary for the motion control of the robot arm includes a robot ID for identifying an individual or a model of the surgical robot, robot configuration information such as number of inks and number of joints, calibration information for when controlling the motion of the robot arm, and the like. Furthermore, the storage circuit may be writable, and information associated with the usage state of the surgical robot 200 may be written from the slave device 120 side. The information associated with the usage state may be a flag indicating the presence or absence of use, a combination of an operation ID for identifying an operation, an operating room ID for identifying an operating room, a date and time on which an operation is performed, and the like.

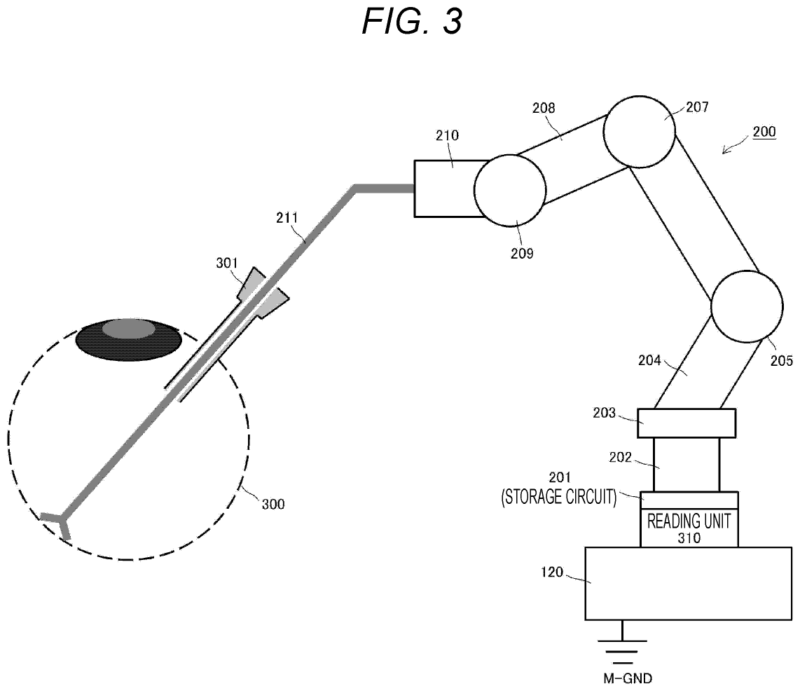

FIG. 3 illustrates a layout of an eyeground operation using the surgical robot 200 illustrated in FIG. 2. An eye lid speculum (not illustrated) is attached to the eyeball 300 so that the eyelid does not close. Then, the trocar 301 is punctured into the surface of the eyeball 300. FIG. 3 illustrates a cross-section of eyeball 300 cut such that trocar 301 passes therethrough. The surgical tool 211 loaded at the distal end of the surgical robot 200 is inserted into the eyeball 300 via one trocar 301.

In the example illustrated in FIG. 3, the surgical robot 200 is assumed to be rigidly fixed to a mechanical ground (M-GND) in a state of being attached to the slave device 120 by the interface unit 201. In addition, the slave device 120 is equipped with a reading unit 310 at an attachment portion of the interface unit 201, and can read information from a storage circuit in the interface unit 201 from the reading unit 310. When the storage circuit is writable, information is written to the storage circuit via the reading unit 310.

Note that a method of accessing the storage circuit by the reading unit 310 is not particularly limited. The access may be an electrical contact system or a non-contact system using an electromagnetic, magnetic, or optical action. In addition, it is assumed that the reading unit 310 includes an installation detection mechanism that detects whether or not the surgical robot 200 is installed.

The slave-side control unit 121 in the slave device 120 can easily set up the surgical robot by reading necessary information from the storage circuit loaded on the surgical robot 200 using the reading unit 310 every time the surgical robot 200 is newly installed.

In addition, the surgical robot 200 is made disposable, and after being used in the operation once, it needs to be reliably discarded so as not to be used again by mistake. In the present embodiment, the interface unit 201 of the surgical robot 200 is loaded with a writable storage circuit to store information associated with the usage state. The information associated with the usage state may be a flag indicating the presence or absence of use, a combination of an operation ID for identifying an operation, an operating room ID for identifying an operating room, a date and time on which an operation is performed, and the like. Then, the slave-side control unit 121 in the slave device 120 accesses the storage circuit of the interface unit 201 through the reading unit 310, confirms that the usage state of the surgical robot 200 is unused, and then starts using the surgical robot 200. In addition, the slave-side control unit 121 writes the usage history of the surgical robot 200 or the information for identifying the used operation in the storage circuit of the interface unit 201 through the reading unit 310.

In such a case, the slave-side control unit 121 can access the storage circuit at the time of setting up the surgical robot, and can start using the surgical robot 200 after confirming that the surgical robot 200 is unused. Then, when the used surgical robot is installed by mistake, the slave-side control unit 121 may issue a warning to urge disposal of the surgical robot and install a new surgical robot, or restrict the operation of the surgical robot.

Note that the operation performed by the slave-side control unit 121 in the above description may be performed by the master-side control unit 111 of the master device 110. Furthermore, the warning described above may be performed on the master device 110 side using the presentation unit 113.

In a case where the surgical robot 200 is assumed to be used in the ophthalmic operation, the movable range may be small, and hence the robot arm becomes small in size, and the number of parts and device cost can be reduced, so that even if the robot arm is made disposable, an increase in operation cost is suppressed, and the burden of disposal work is reduced.

### D. Replacement of Surgical Tool

It is assumed that the surgical tool mounted to the surgical robot 200 is replaced in one operation. In the surgical system 100, it is necessary to acquire and set up information necessary for operating the surgical tool through the surgical robot every time the surgical tool mounted to the surgical robot 200 is replaced. In the present embodiment, the surgical tool itself may be loaded with a storage circuit that stores necessary information, and the surgical robot 200 may transmit information read from the storage circuit in the surgical tool being mounted to the surgical system 100 side. Therefore, the surgical system 100 can easily set up the surgical robot 200 and the surgical tool and immediately start the surgical tool operation. Note that the information stored in the storage circuit in the surgical tool includes a surgical tool ID for identifying the individual or the model of the surgical tool, calibration information when operating the surgical tool (e.g., when opening/closing the forceps), and the like.

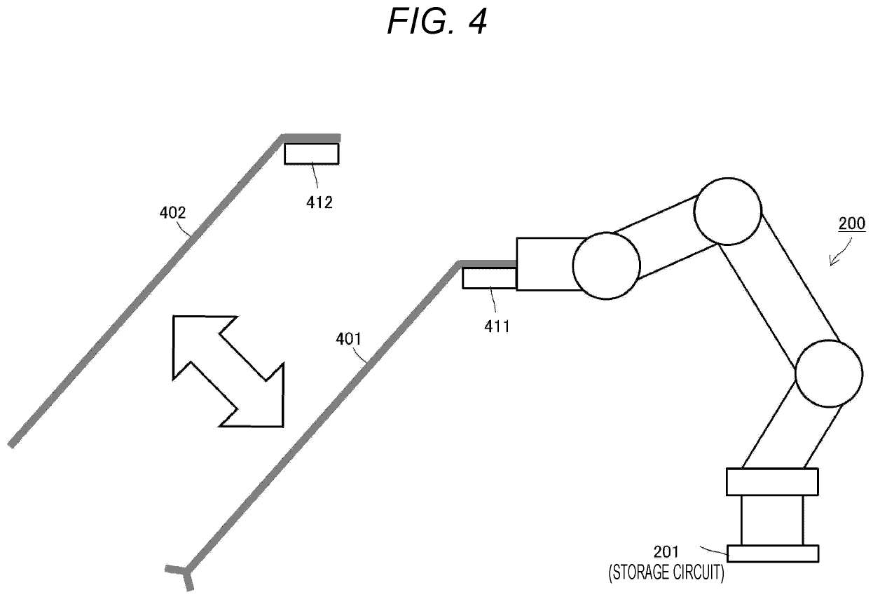

FIG. 4 illustrates a state in which a surgical tool 401 mounted to the surgical robot 200 is replaced with a surgical tool 402. A storage circuit 411 is loaded in the surgical tool 401, and a storage circuit 412 is loaded in the surgical tool 402. Therefore, when the surgical tool 402 is newly mounted to the surgical robot 200, the slave-side control unit 121 in the slave device 120 can immediately read necessary information from the storage circuit 412 of the surgical tool 402, set up the surgical tool 412, and immediately start the surgical tool operation.

In addition, it is assumed that the surgical tool is once attached to the surgical robot and then detached therefrom in one operation, and then used again in the same surgical robot (or another robot). This is because there is no problem such as infection of bacteria if the patients are the same. Therefore, each of the surgical tools 401 and 402 is loaded with the writable storage circuits 411 and 412, respectively, and stores, in the storage circuit, the presence or absence of use, a patient ID for specifying a patient who has used the surgical tool, and an operation ID (or may be a combination of an operating room ID for identifying an operating room and a date and time on which an operation is performed) for identifying operation.

In such a case, the slave-side control unit 121 in the slave device 120 can read the information of the presence or absence of use, the patient ID, or the operation ID from the storage circuit 411 or 412 of the surgical tool 401 or 402 being mounted through the reading unit 310, and check whether or not the surgical tool is unused and whether or not the surgical tool is a surgical tool to be used again in one operation. Then, when the surgical tool used in the operation of another patient is mounted to the surgical robot by mistake, the surgical system 100 may issue a warning to urge replacement with a new surgical tool or restrict the operation of the surgical robot.

Note that the operation performed by the slave-side control unit 121 in the above description may be performed by the master-side control unit 111 of the master device 110. Furthermore, the warning described above may be performed on the master device 110 side using the presentation unit 113.

### E. Authentication Process

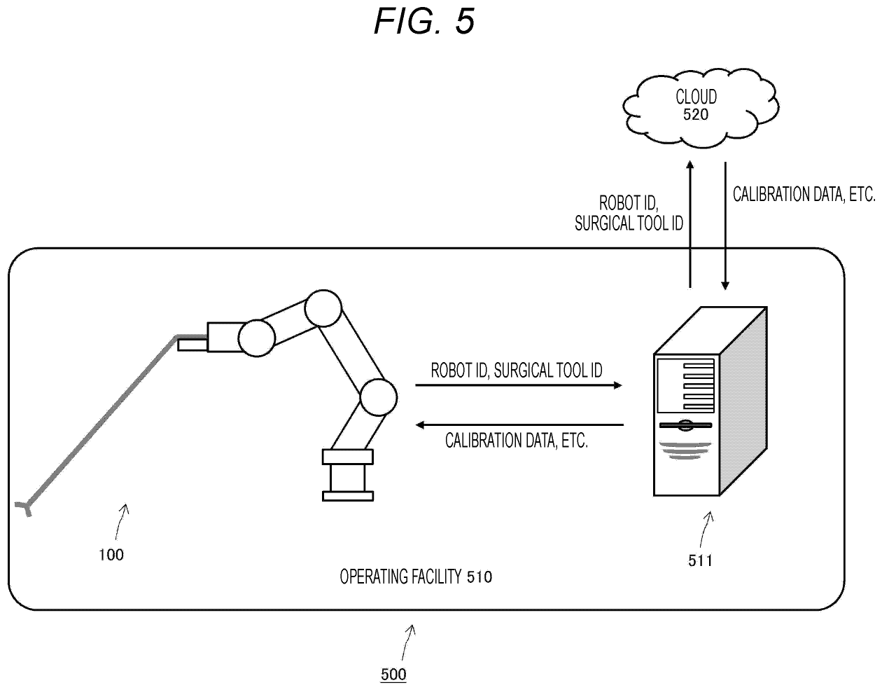

FIG. 5 schematically illustrates a configuration example of a surgical robot 200 attached to the surgical system 100 and an authentication system 500 that performs an authentication process of a surgical tool mounted to the surgical robot 200. FIG. 5 illustrates the authentication system 500 that performs an authentication process using a cloud.

In an operating facility 510 such as a hospital, the surgical system 100 and an authentication server 511 that performs the authentication process of disposable components such as a surgical robot and a surgical tool used in the surgical system 100 are arranged.

Here, it is assumed that a surgical robot is installed in the surgical system 100 and a surgical tool is mounted to the surgical robot. Then, it is assumed that the surgical system 100 can read the robot ID from the storage circuit loaded on the surgical robot and can read the surgical tool ID from the storage circuit loaded on the surgical tool mounted on the surgical robot.

The surgical system 100 transfers the robot ID and the surgical tool ID read from the surgical robot and the surgical tool to the authentication server 511.

The authentication server 511 inquires the cloud 520 of the robot ID and the surgical tool ID acquired from the surgical system 100, and performs the authentication process of the surgical robot and the surgical tool used in the surgical system 100, the authentication server being interposed between the cloud 520 and the surgical system 100.

In the authentication process, for example, whether the surgical robot or the surgical tool is a genuine product or an authorized product, and whether the operation of these components can be guaranteed are confirmed on the basis of the ID information. Then, the authentication server 511 notifies the surgical system 100 of the result of the authentication process.

In a case where the surgical system 100 receives the notification indicating that the authentication has failed from the authentication server 511, the surgical system 100 does not permit the start of the surgical operation using the surgical robot and the surgical tool, and notifies the user that the authentication has failed. For example, the user may be notified using the presentation unit 113. For example, in a case where it is found that the surgical robot or the surgical tool is not a genuine product or an authorized product, a warning urging the user to replace these unauthorized parts may be output from the presentation unit 113.

On the other hand, in a case where the authentication is successful, the surgical system 100 can permit the use of the surgical robot and the surgical tool and start the surgical operation (e.g., ophthalmic operation).

Note that, in the above sections B to D, description has been made on the fact that the usage state and the calibration information are stored in the storage circuit loaded on the surgical robot or the surgical tool, but the usage state and the calibration information may be managed by being associated with each ID of the surgical robot and the surgical tool on the cloud 520. In this case, when the authentication of the robot ID and the surgical tool ID inquired from the authentication server 511 is successful, the cloud 520 further checks the usage state of the robot ID and the surgical tool ID, and finally returns a result of successful authentication to the authentication server 511 only in a case where it is confirmed that the robot and the surgical tool are unused.

In addition, in a case where the authentication is successful in the cloud 520, calibration data, robot configuration information, and surgical tool configuration information associated with the robot ID and the surgical tool ID may also be returned to the authentication server 511. Then, the surgical system 100 (e.g., the slave-side control unit 121 in the slave device 120) may set up the surgical robot and the surgical tool installed in the surgical system 100 on the basis of the calibration data, the robot configuration information, and the surgical tool configuration information provided from the cloud 511.

### F. Processing Operation of Surgical System

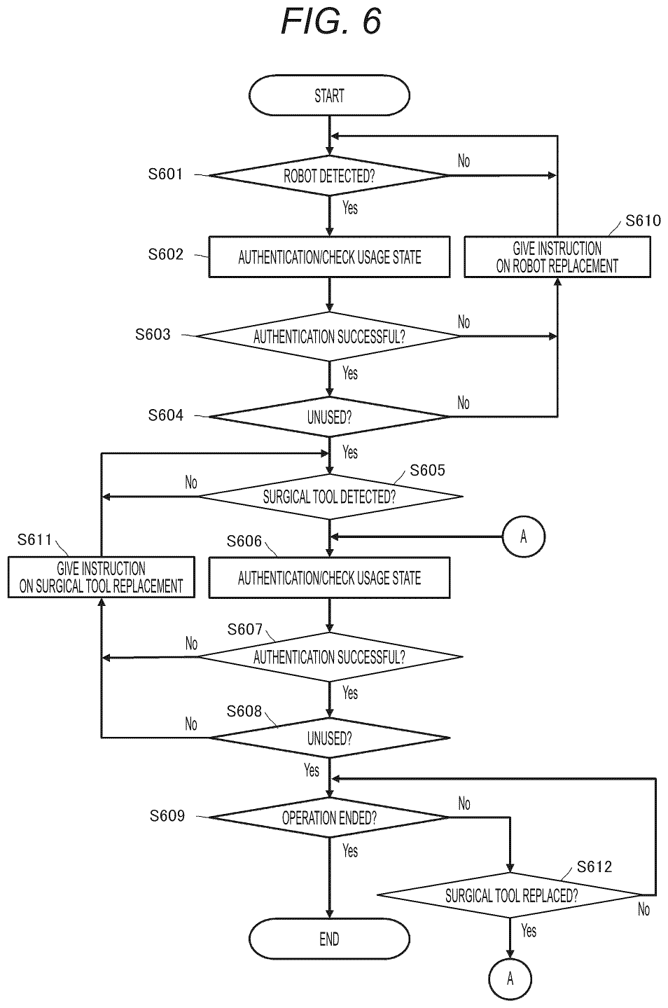

FIG. 6 is a flowchart illustrating a processing procedure executed in the surgical system 100 using a disposable surgical robot and surgical tool. This processing procedure is mainly executed by the slave-side control unit 121 on the slave device 120 side, for example, but of course, necessary information can be transferred from the slave device 120 to the master device 110 and executed by the master-side control unit 111 in the master device 110.

When detecting that a surgical robot is newly installed as the slave robot 122 (Yes in step S601), the slave-side control unit 121 performs an authentication process of and checks a usage state of the surgical robot (step S602).

The authentication process of the surgical robot is, for example, as described in section E above. In a case where the authentication process fails (No in step S603), the user is instructed to replace the surgical robot (step S610), and the process returns to step S601. Furthermore, in a case where the detected surgical robot is already used (No in step S604), the user is instructed to replace the surgical robot with an unused surgical robot (step S610), and the process returns to step S601. Note that the replacement instruction of the surgical robot may be made using the presentation unit 113 on the master device 110 side.

On the other hand, in a case where the authentication process of the newly installed surgical robot is successful and it can be confirmed that the surgical robot is in the unused state (Yes in steps S603 and S604), the slave-side control unit 121 checks whether or not a surgical tool is mounted to the distal end of the surgical robot (step S605).

When detecting that the surgical tool is mounted to the surgical robot (Yes in step S605), the slave-side control unit 121 performs the authentication process of the relevant surgical tool and the check of the usage state (step S606).

The authentication process of the surgical robot is, for example, as described in section E above. In a case where the authentication process fails (No in step S607), the user is instructed to replace the surgical tool (step S611), and the process returns to step S605. Furthermore, in a case where the surgical tool mounted to the surgical robot is already used (No in step S608), the user is instructed to replace the surgical tool with an unused surgical tool (step S611), and the process returns to step S605. Note that the replacement instruction of the surgical tool may be made using the presentation unit 113 on the master device 110 side.

On the other hand, in a case where the authentication process of the surgical tool mounted to the surgical robot is successful and it can be confirmed that the surgical tool is in the unused state (Yes in steps S607 and S608), operation using the surgical robot 100 can be started in the surgical system 100.

Until the operation ends (No in step S609), the slave-side control unit 121 continues to monitor whether or not the surgical tool mounted to the surgical robot is replaced (step S612). When detecting that the surgical tool mounted to the surgical robot is replaced (Yes in step S612), the slave-side control unit 121 returns to step S606 and repeatedly performs the authentication process of the replaced surgical tool and the check of the usage state.

### G. Implementation Example of Surgical Robot

In section G, a configuration example of a disposable surgical robot configured using a flexible electric circuit board having low rigidity and flexibility will be described.

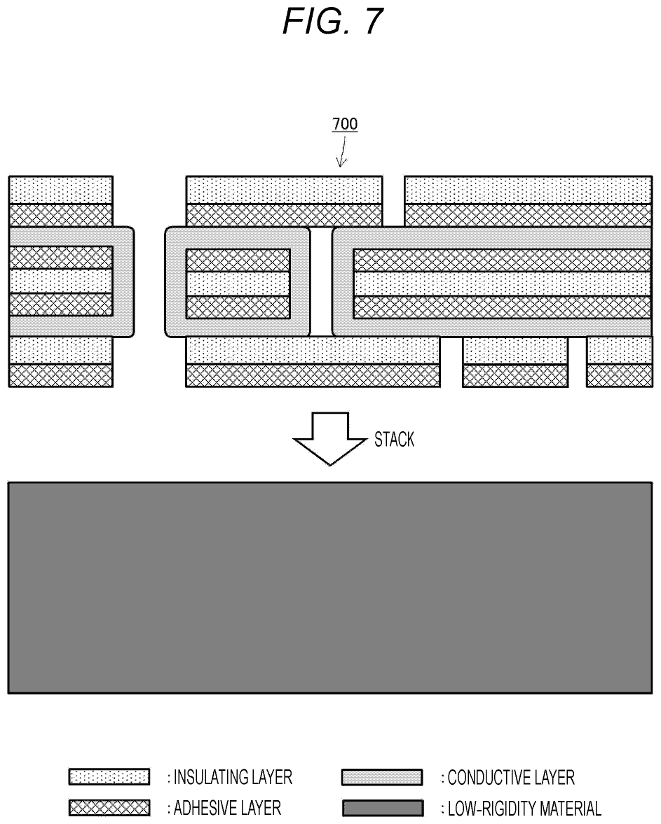

FIG. 7 is a diagram illustrating a cross-sectional configuration example of an electric circuit board 700 applied to a multi-link structure. As can be seen from the drawing, the electric circuit board 700 includes an insulating layer including a high-electron polymer or polyimide and a conductive layer formed by vapor depositing a metal such as copper or aluminum, and is a multilayer structure in which a plurality of sets of an insulating layer and a conductive layer is joined by an adhesive layer. A method for manufacturing the electric circuit board 700 having such a multilayer structure is not particularly limited. For example, there is also a method of adhering the insulating layer and the conductive layer by providing an adhesive layer on the conductive layer prepared in advance. Finally, the multilayer structure including the insulating layer, the conductive layer, and the adhesive layer is covered with a low rigidity material including polyimide or the like, so that the electric circuit board 700 having low rigidity and flexibility is realized. The electric circuit board 700 may be the same as a general flexible printed circuit (FPC).

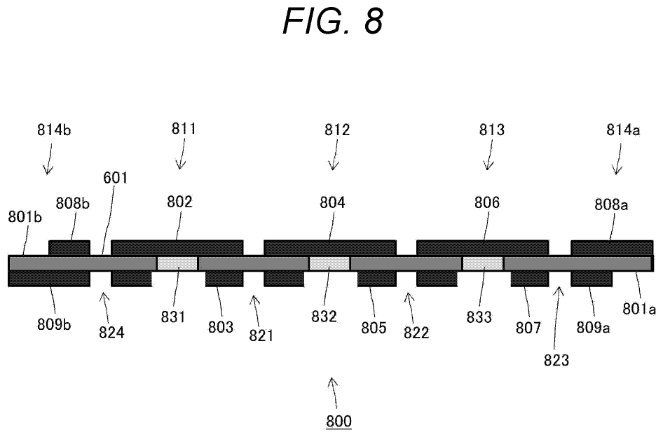

FIG. 8 illustrates a configuration example of an open link structure 800 configured using an FPC. In the open link structure 800, the low rigidity FPC 801 is disposed at the center, a pair of strong rigidity portions 802 and 803 are joined to the front and back surfaces of the FPC 801 to form a rigid link 811, a pair of strong rigidity portions 804 and 805 are joined to the front and back surfaces of the FPC 801 to form a rigid link 812, a pair of strong rigidity portions 806 and 807 are joined to the front and back surfaces of the FPC 801 to form a rigid link 813, a pair of strong rigidity portions 808a and 809a are joined to the front and back surfaces of the FPC 801 to form a rigid link 814a, and a pair of strong rigidity portions 808b and 809b are joined to the front and back surfaces of the FPC 801 to form a rigid link 814b. The link 811 and the link 812, the link 812 and the link 813, the link 813 and the link 814a, and the link 814b and the link 811 constitute hinges 821, 822, 823, and 824 connected by the FPC 801.

In the open link structure 800, the strong rigid portion 803 has an opening in the center, and the conductive layer of the FPC 801 is exposed to the outside through the opening so that the link 811 has an electrode pad 831 for electrical connection or signal extraction, the strong rigid portion 805 has an opening in the center, and the conductive layer of the FPC 801 is exposed to the outside through the opening so that the link 812 has an electrode pad 832 for electrical connection or signal extraction, and the strong rigid portion 807 has an opening in the center, and the conductive layer of the FPC 801 is exposed to the outside through the opening so that the link 813 has an electrode pad 633 for electrical connection or signal extraction. Furthermore, the electrode pads 801a and 801b are provided at the respective ends of the links 814a and 814b at both ends of the open link structure 800.

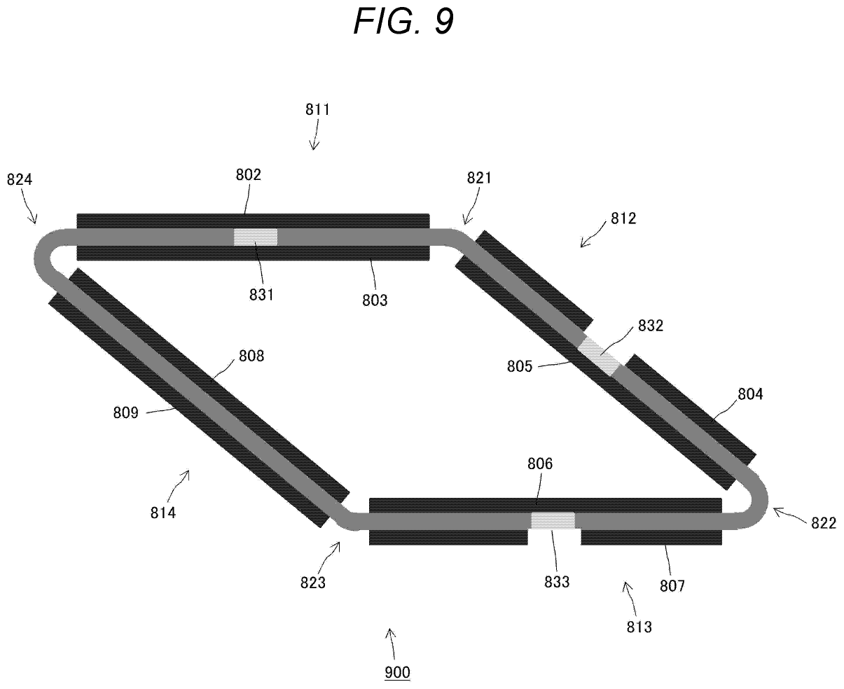

FIG. 9 illustrates an example of a closed link structure 900 configured using an FPC. The illustrated closed link structure 900 forms a closed link structure by bending the FPC 801 constituting the open link structure 800 illustrated in FIG. 8 and joining the electrode pads 801a and 801b provided on the links 814a and 814b at both ends. The joined links 814a and 814b are newly defined as a link 814.

In the closed link structure 900, the lengths of the opposing link 811 and the link 813 and the lengths of the link 812 and the link 814 are equal to each other, and thus a parallel link mechanism (or four-joint link mechanism) can be configured. Therefore, when the prime moving link moves, the driven link makes the same movement, and the angle of the opposing links is always maintained.

Each of the hinge portions 821, 822, 823, and 824 is a joint configured only by the FPC. Since the conductive layer of the FPC passes through the rotation axis in each of the hinge portions 821, 822, 823, and 824, it can be said as the wiring structure passing through the hinge portion. Therefore, even if the rotational operation occurs between the links, stress such as tension and compression force affecting conductivity can be suppressed to be low, and thus, an adverse effect on control performance and a risk of cutting the wiring are extremely low.

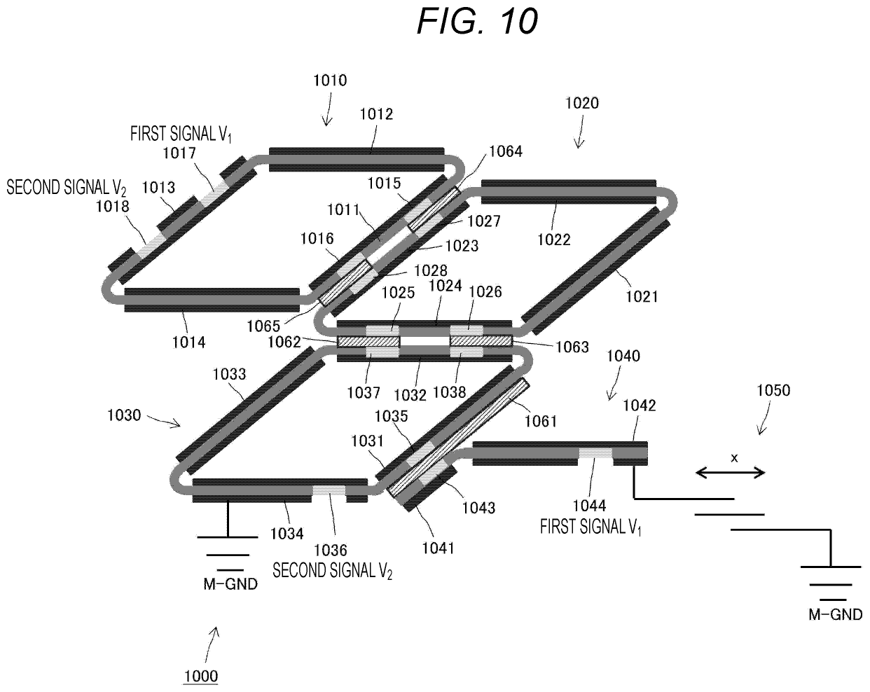

FIG. 10 illustrates a configuration example of the surgical robot 1000 in which a plurality of closed link structures using the FPC as illustrated in FIG. 9 is coupled. In the surgical robot 1000, the closed link structure 1010, the closed link structure 1020, and the closed link structure 1030 are coupled in this order from the distal end. Furthermore, an open link structure 1040 having a linear motion mechanism is connected to the closed link structure 1030. The specific configurations of each of the closed link structures 1010 to 1030 and the open link structure 1040 are similar to those illustrated in FIGS. 7 and 8, and thus the detailed description thereof will be omitted here.

One link 1034 of the closed link structure 1030 on the proximal end side serves as a mechanical ground (or the fixed link). The link 1041 of the open link structure 1040 is connected to the link 1031 hinged to one end of the link 1034. Furthermore, the link 1042 of the open link structure 1040 can be displaced in the horizontal direction in the plane of drawing (or x direction) by the linear motion actuator 1050 having one end serving as a mechanical ground. Therefore, the link 1031 is a prime moving link. The link 1033 facing the link 1031 is a driven link, and the other links 1032 are intermediate links.

The open link structure 1040 includes an electrode pad 1043 at one area of the link 1041 and an electrode pad 1044 at one area of the link 1043. The electrode pad 1044 is used to input and output the first signal V₁, and the electrode pad 1043 is used to transmit the first signal V₁ to and from the closed link structure 1030 side.

The link 1031 of the closed link structure 1030 has one electrode pad 1035 at a position facing the electrode pad 1043. Then, the link 1041 of the open link structure 1040 is fixed to the link 1031 on the closed link structure 1030 side while ensuring the conductivity of the electrode pad 1044 and the electrode pad 1035 through the conductive joining portion 961. Therefore, the closed link structure 1030 is capable of transmitting the first signal V₁ to and from the open link structure 1040. In addition, the closed link structure 1030 has an electrode pad 1036 at one area of the link 1034. The electrode pad 1036 is used to input and output the second signal V₂.

The closed link structure 1030 includes electrode pads 1037 and 1038 for a first signal and a second signal, respectively, at two areas of the link 1032. Furthermore, the link 1024 coupled to the link 1032 on the closed link structure 1020 side has two electrode pads 1025 and 1026 at positions facing the electrode pads 1037 and 1038, respectively. The link 1024 is fixed to the link 1032 while ensuring conductivity between the electrode pad 1025 and the electrode pad 1037 and between the electrode pad 1026 and the electrode pad 1038 through the joining portions 1062 and 1063 having conductivity, respectively. Therefore, the first signal V₁ and the second signal V₂ can be transmitted between the closed link structure 1030 and the closed link structure 1020.

The closed link structure 1020 includes electrode pads 1027 and 1028 for the first signal V₁ and the second signal V₂, respectively, at two areas of the link 1023. Furthermore, the link 1011 coupled to the link 1023 on the closed link structure 1010 side has two electrode pads 1015 and 1016 at positions facing the electrode pads 1027 and 1028, respectively. The link 1011 is fixed to the link 1022 while ensuring conductivity between the electrode pad 1015 and the electrode pad 1027 and between the electrode pad 1016 and the electrode pad 1028 through the joining portions 1064 and 1065 having conductivity, respectively. Therefore, the first signal V₁ and the second signal V₂ can be transmitted between the closed link structure 1020 and the closed link structure 1010.

A link 1013 of the closed link structure 1011 corresponds to a link at a distal end of the surgical robot 1000, and constitutes a mounting portion of an end effector (not illustrated in FIG. 9) including a surgical tool such as forceps. Then, the electrode pads 1017 and 1018 for the first signal V₁ and the second signal V₂ are provided at two areas of the link 1013, respectively. Therefore, the surgical robot 1000 can transmit the first signal V₁ and the second signal V₂ to and from an end effector (not illustrated in FIG. 10) mounted to the distal end.

The surgical tool used by being mounted to the surgical robot 1000 holds a storage circuit that stores, for example, a surgical tool ID for identifying the type, specification, performance, or individual information of the surgical tool, authentication information for determining whether or not to use the surgical tool by the authentication system 500, calibration data when operating the surgical tool, and the like. Then, the surgical robot 1000 can access the surgical tool through the electrical interface including the electrode pads 1017 and 1018 at the distal end, read the surgical tool ID from the surgical tool, and transmit the corresponding authentication information, calibration data, and the like to the storage circuit in the surgical tool.

In the surgical robot 1000 according to the present embodiment, a signal lines used for transmission of the first signal V₁ and the second signal V₂ have a wiring structure passing through the hinge. Therefore, even if the surgical robot 1000 operates and a rotational operation occurs between the links, stress such as tension and compression force affecting conductivity can be suppressed to be low, so that adverse effects on control performance and a risk of cutting the wiring are extremely low.

On the signal transmission path, a control signal and power to the surgical tool which is the end effector, a signal of information read from a memory in the surgical tool, and the like are transmitted. Note that FIG. 10 illustrates an example in which the surgical robot 1000 includes a signal transmission path of 2 bits of the first signal V₁ and the second signal V₂, but the bit width of the signal transmission path can be easily extended to 3 bits or more.

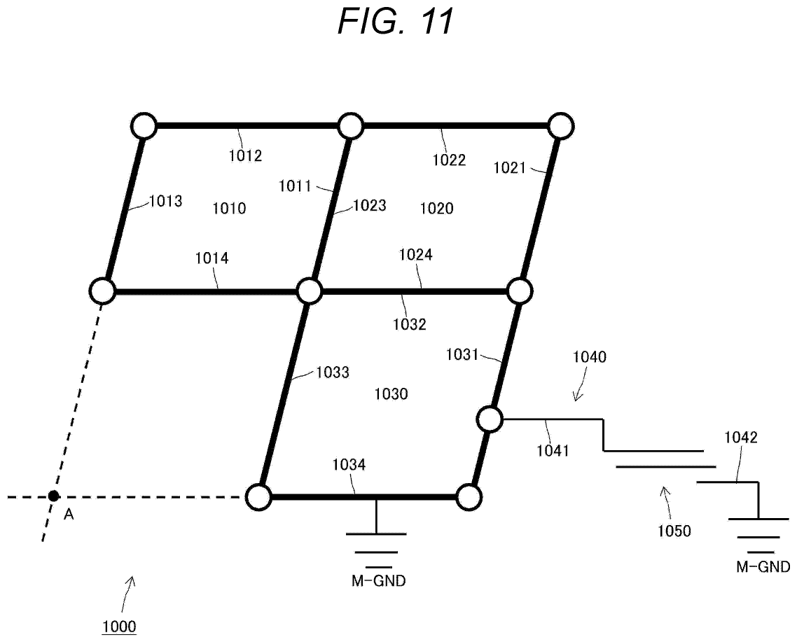

FIG. 11 illustrates a configuration of a degree of freedom of the surgical robot 1000 illustrated in FIG. 10. However, in FIG. 11, a high rigidity link is drawn with a thick line, and a joint for hinge-coupling the links is indicated by a circle coaxial with the rotation axis.

The surgical robot 1000 is configured by coupling three closed link structures 1010, 1020, and 1030 in order from the distal end. One link 1034 of the closed link structure 1030 on a proximal end side serves as a mechanical ground (or a fixed link).

The closed link structure 1010 is a four-joint link mechanism including four links 1011 to 1014, and the lengths of the opposing links are equal to each other. The closed link structure 1020 is a four-joint link mechanism including four links 1021 to 1024, and the lengths of the opposing links are equal to each other. Furthermore, the closed link structure 1030 is a four-joint link mechanism including four links 1031 to 1034, and the lengths of the opposing links are equal to each other. However, the link 1012 and the link 1022, the link 1014 and the link 1024, and the link 1021 and the link 1031 are linearly coupled to operate as one link. In addition, the link 1011 and the link 1023 that join the closed link structure 1010 and the closed link structure 1020 are integrated to operate as one link, and the link 1024 and the link 1032 that join the closed link structure 1020 and the closed link structure 1030 are integrated to operate as one link.

The open link structure 1040 is coupled to a link 1031 hinged to one end of the link 1034. The open link structure 1040 includes two links 1041 and 1042 linearly coupled via a linear motion actuator 1050. One link 1042 has one end rigidly fixed to the mechanical ground. When the linear motion actuator 1050 is operated and the link 1041 is displaced in the horizontal direction (or x direction) in the plane of drawing, the link 1031 rotates about the coupling point with the link 1034. Therefore, the closed link structure 1030 operates such that the link 1031 is a prime driving link, the link 1033 facing the link 1031 is a driven link, and the other links 1032 are intermediate links, and the angle of the opposing links is maintained. This operation is then transferred to the adjacent closed link structure 1020 and further to the closed link structure 1010 adjacent to the closed link structure 1020.

A link 1013 of the closed link structure 1011 corresponds to a link at a distal end of the surgical robot 1000, and constitutes a mounting portion of an end effector (not illustrated in FIG. 11) including a surgical tool such as forceps. In a case where the surgical tool mounted to the distal end of the surgical robot 1000 is operated to perform a surgical operation, it is necessary to perform, for minimum invasiveness, the operation with a load as small as possible on the vicinity of a trocar into which the surgical tool is inserted, so that it is ideal to cause the surgical tool to pivot using the trocar insertion point as a fulcrum (or with the trocar insertion point fixed) to make an impulse generated at the trocar insertion point equal to zero.

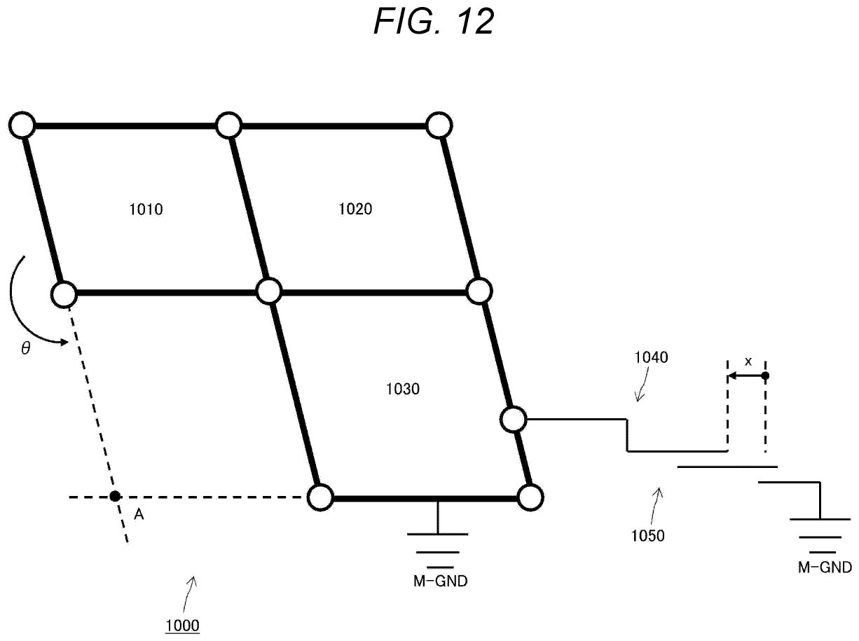

FIG. 12 illustrates a state in which the link 1031, which is the prime moving link of the closed link structure 1030, is rotated by an angle θ in a counterclockwise direction in the plane of drawing via the open link structure 1040 by displacing the linear motion actuator 1050 in the x direction. Assuming that each link of the other closed link structure 1020 and the closed link structure 1010 is kept in parallel relationship with a corresponding link of the closed link structure 1030, the axis of the link (fixed link) 1034 of the closed link structure 1030 and the axis of the link 1013, to which the surgical tool is mounted, of the closed link structure 1010 at the distal end also intersect at point A. That is, the intersection point A is a fixed point. Therefore, by setting the intersection point A to be the trocar insertion point, the surgical tool mounted to the link 1013 pivots at the inserting portion, and minimally invasive operation can be realized.

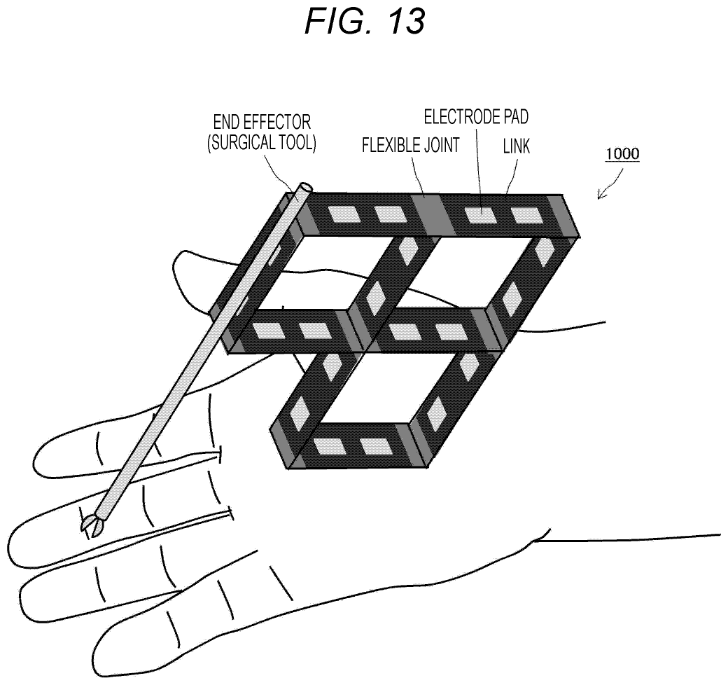

In FIG. 10, for the sake of convenience of description, the surgical robot 1000 is illustrated as a plan view as viewed from the side, and each link is drawn like a wire rod. In practice, the link is a rigid body having a constant width because it uses the FPC as a base material. FIG. 13 illustrates an example of a three-dimensional image of the surgical robot 1000 configured by a closed link structure using the FPC.

An end effector including a surgical tool such as forceps is attached to a link at a distal end of the surgical robot 1000. Since the surgical robot 1000 is basically configured using an FPC, it is easy to manufacture the surgical robot in a small size for ophthalmic operation. For example, the surgical robot 1000 that is small enough to be placed on a palm can be manufactured at a relatively low cost and can be made disposable. In addition, the surgical tool can be similarly made to be disposable.

Note that the multilink structure using the electric circuit board as described in section G is merely an example, and a disposable surgical robot can be manufactured without using such a multilink structure.

### H. Effects

Finally, effects brought by the present disclosure will be summarized.

(1) Since the surgical robot and the surgical tool mounted to the surgical robot are respectively loaded with a storage circuit that stores information required at the time of setup, even if the surgical robot and the surgical tool are made disposable and replaced for each operation, the setup can be easily performed by accessing the storage circuit of the surgical robot and the surgical tool from the system side and acquiring the necessary information.

(2) The residual protein can be reduced and the risk of complications can be avoided by making the surgical robot and the surgical tool disposable and replacing them for each operation.

(3) The residual protein can be reduced and the risk of complications can be avoided by making the surgical robot and the surgical tool disposable and replacing them for each operation.

(4) The surgical drape becomes unnecessary by making the surgical robot and the surgical tool disposable and replacing them for each operation, and thus the setup work for the drape is not required, and problems such as contact with the drape, collision with another surgical tool, and visual field obstruction of the microscope due to the drape during the operation do not arise.

### INDUSTRIAL APPLICABILITY

The present disclosure has been described in detail above with reference to the specific embodiments. However, it is obvious that those skilled in the art can make modifications and substitutions of the embodiments without departing from the gist of the present disclosure.

In the present specification, the embodiment in which the present disclosure is mainly applied to ophthalmic operation has been mainly described, but the gist of the present disclosure is not limited thereto. Even when the present disclosure is applied to various other types of surgical operations, the entire surgical robot can be made disposable, and the setup work of the disposable surgical robot can be facilitated.

In short, the present disclosure has been described in the form of exemplification, and thus the contents described herein should not be construed in a limited manner. To determine the gist of the present disclosure, the scope of claims should be taken into consideration.

Note that the present disclosure can have the following configurations.

(1) A surgical robot equipped with:
a robot arm to which a surgical tool is mounted; and
a storage circuit that stores information associated with motion control of the robot arm.

(2) The surgical robot described in (1) above, in which
the storage circuit stores at least one of a robot ID for identifying an individual or a model of the surgical robot, configuration information of the robot arm, or calibration information for when controlling the motion of the robot arm.

(3) The surgical robot described in any one of (1) and (2) above, in which
the storage circuit is writable and stores information for identifying presence or absence of use or operation used.

(4) The surgical robot described in any one of (1) to (3) above, in which
the surgical tool is loaded with a storage circuit, and
performs reading or writing on the storage device loaded in the surgical tool.

(5) The surgical robot described in (4) above, in which
the storage circuit loaded in the surgical tool stores at least one of a surgical tool ID for identifying an individual or a model of the surgical tool or calibration information for when operating the surgical tool.

(6) The surgical robot described in any one of (4) or (5) above, in which
the storage circuit loaded in the surgical tool is writable and stores at least one of presence or absence of use, information for specifying a patient to whom the surgical tool is used, or information for identifying operation using the surgical tool.

(7) The surgical robot described in any one of (1) to (6) above, in which
the storage circuit is accessible from the outside of the surgical robot by a predetermined system.

(7-1) The surgical robot described in (7) above, in which
the predetermined system includes at least one of an electrical contact system and a non-contact system using an electromagnetic, magnetic, or optical action.

(8) The surgical robot described in any one of (1) to (7) above, in which
the surgical tool is used for ophthalmic operation.

(9) A surgical system including:
a surgical robot with a robot arm to which a surgical tool is mounted and a storage circuit that stores information associated with motion control of the robot arm; and
a control unit that controls the surgical robot on the basis of the information read from the storage circuit.

(10) The surgical system described in (9), in which
the control unit performs an authentication process of the surgical tool or the surgical robot on the basis of identification information of the surgical tool read from the storage circuit loaded in the surgical tool or identification information of the surgical robot read from the storage circuit.

(11) The surgical system described in any one of (9) or (10), in which
the control unit determines availability of the surgical tool or the surgical robot on the basis of information associated with a usage state of the surgical tool read from the storage circuit loaded in the surgical tool or information associated with a usage state of the surgical robot read from the storage circuit.

(12) The surgical system described in any one of (9) to (11), in which
the control unit controls an operation of the surgical tool by the surgical robot on the basis of calibration information of the surgical tool read from the storage circuit loaded in the surgical tool.

### REFERENCE SIGNS LIST

100 Surgical system
110 Master device
111 Master-side control unit
112 Operation UI unit
113 Presentation unit
114 Master-side communication unit
120 Slave device
121 Slave-side control unit
122 Slave robot
123 Sensor unit
124 Slave-side communication unit
130 Transmission path
200 Surgical robot
201 Interface unit
202 Link
203, 205, 207, 209 Joint
204, 206, 208, 210 Link
211 Surgical tool
301 Trocar

1. A surgical robot comprising:
a robot arm to which a surgical tool is mounted; and
a storage circuit that stores information associated with motion control of the robot arm.

2. The surgical robot according to claim 1, wherein
the storage circuit stores at least one of a robot ID for identifying an individual or a model of the surgical robot, configuration information of the robot arm, or calibration information for when controlling the motion of the robot arm.

3. The surgical robot according to claim 1, wherein
the storage circuit is writable and stores information for identifying presence or absence of use or operation used.

4. The surgical robot according to claim 1, wherein
the surgical tool is loaded with a storage circuit, and
performs reading or writing on the storage device loaded in the surgical tool.

5. The surgical robot according to claim 4, wherein
the storage circuit loaded in the surgical tool stores at least one of a surgical tool ID for identifying an individual or a model of the surgical tool or calibration information for when operating the surgical tool.

6. The surgical robot according to claim 4, wherein
the storage circuit loaded in the surgical tool is writable and stores at least one of presence or absence of use, information for specifying a patient to whom the surgical tool is used, or information for identifying operation using the surgical tool.

7. The surgical robot according to claim 1, wherein
the storage circuit is accessible from the outside of the surgical robot by a predetermined system.

8. The surgical robot according to claim 1, wherein
the surgical tool is used for ophthalmic operation.

9. A surgical system comprising:
a surgical robot including a robot arm to which a surgical tool is mounted and a storage circuit that stores information associated with motion control of the robot arm; and
a control unit that controls the surgical robot on a basis of the information read from the storage circuit.

10. The surgical system according to claim 9, wherein
the control unit performs an authentication process of the surgical tool or the surgical robot on a basis of identification information of the surgical tool read from the storage circuit loaded in the surgical tool or identification information of the surgical robot read from the storage circuit.

11. The surgical system according to claim 9, wherein
the control unit determines availability of the surgical tool or the surgical robot on a basis of information associated with a usage state of the surgical tool read from the storage circuit loaded in the surgical tool or information associated with a usage state of the surgical robot read from the storage circuit.

12. The surgical system according to claim 9, wherein
the control unit controls an operation of the surgical tool by the surgical robot on a basis of calibration information of the surgical tool read from the storage circuit loaded in the surgical tool.
